(19) United States
(12) Patent Application Publication
(10) Pub. No.: US 2005/0203179 A1
(43) Pub. Date: Sep. 15, 2005

(54) Use of hydroxydiphenyl ether derivatives as arylsulfatase-inhibitors in deodorants and antiperspirants

(76) Inventors: Bernhard Banowski, Dusseldorf (DE); Armin Wadle, Erkrath (DE); Petra Siegert, Haan (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/511,015

(22) PCT Filed: Apr. 7, 2003
(86) PCT No.: PCT/EP03/03603

§ 371 Date: Apr. 22, 2005

(30) Foreign Application Priority Data
Apr. 12, 2002 (DE) 102 16 368.5

(51) Int. Cl. A61K 31/235; A61K 31/192; A61K 31/075

(52) U.S. Cl. 514/532; 514/718; 514/679; 514/571

(57) ABSTRACT

The invention relates to the use of selected hydroxydiphenyl ether derivatives as arylsulfatase-inhibiting substances in a cosmetic deodorant or antiperspirant composition for reducing body odor caused by decomposition of steroid esters.

The invention relates to the use of selected arylsulfatase-inhibiting substances in a cosmetic deodorant or antiperspirant composition for reducing body odour caused by the decomposition of steroid esters.

Apocrine sweat is a complex mixture comprising inter alia steroids, cholesterol and other fats and also about 10% proteins. The decomposition products of apocrine sweat, which make a substantial contribution to body odour, especially to axillary body odour, can be divided into two classes: on the one hand short-chain fatty acids, especially C₄-C₁₀fatty acids, which may be linear, branched, saturated or unsaturated, and on the other hand various steroid hormones and metabolites thereof. For example, typical body odour, especially in men, involves the metabolites of androgens, especially androstenol (5α-androst-16-en-3β-ol, 5α-androst-16-en-3α-ol) and androstenone (5α-androst-16-en-3-one). Steroids themselves are not water-soluble. In order that they can be transported away with the body fluids they are usually in sulfate or glucuronide form. On the skin, such steroid esters are cleaved to form the volatile free steroids by hydrolytic enzymes of skin bacteria, especially coryneform bacteria. In principle, all bacterial exoesterases are capable of such action, but the enzyme arylsulfatase especially so.

The deodorant compositions that are active according to the invention are capable of intervening at that point and of inhibiting the activity of the bacterial exoesterases. In that respect they differ from the purely bacteriostatic or bactericidal compositions of the prior art, which can have the disadvantage of adversely affecting the natural microflora of the skin.

The control of steroidally caused body odour through the inhibition of arylsulfatase is known in the prior art, for example from the specifications U.S. Pat. No. 5,643,559 and U.S. Pat. No. 5,676,937, but those documents disclose only a small number of arylsulfatase-inhibiting active ingredients.

Non-halogenated hydroxydiphenyl ethers are known as antimicrobial active ingredients in the prior art from the document EP 1 053 989 A2.

The aim of the present invention was to identify further arylsulfatase-inhibiting active ingredients in order to provide greater variability, flexibility and skin compatibility in the formulation of cosmetic deodorants. The identification of known cosmetic active ingredients as arylsulfatase-inhibitors also makes it possible to reduce the dosage of such active ingredients. The enzyme-inhibiting action is frequently exhibited even at low active ingredient concentrations at which no bacteriostatic or bactericidal action is yet found. It has been found, surprisingly, that the use of arylsulfatase-inhibitors in deodorants is especially suitable for inhibiting the development of body odour in men, the person skilled in the art being able on the basis of his general technical knowledge to match the amount and/or nature of the active ingredients in the deodorant composition gender-specifically to the user group in question.

The present invention relates to the use of at least one arylsulfatase-inhibiting substance selected from hydroxydiphenyl ethers of fgeneral formula 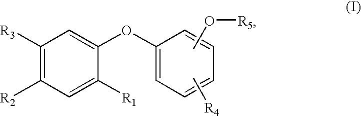
R₁, R₂ and R₃ independently from each other are hydrogen; hydroxy; C₁-C₂₀alkyl; hydroxy-substituted C₁C₂₀alkyl; C₅-C₇cycloalkyl,; C₁-C₂₀alkoxy; C₁-C₆alkylcarbonyl; phenyl; or phenyl-C₁-C₃alkyl;
R₄ hydrogen, C₁-C₂₀alkyl; hydroxy-substitute C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆-alkyl-carbonyl; C₁-C₃alkylcarbonyl-C₁-C₃alkyl; phenyl; or phenyl-C₁-C₃alkyl; and
R₅ is hydrogen; C₁-C₂₀alkoxy; or C₁-C₆alkylcarbonyl.

A C₁-C₂₀alkyl group is to be understood as being a straight-chain or branched alkyl group, e.g. methyl, ethyl, n-propyl, isopropyl, n-butyl, sec-butyl, tert-butyl, pentyl, isopentyl, tert-pentyl, hexyl, cyclohexyl, heptyl, octyl, isooctyl, nonyl, decyl and the like.

A C₁-C₂₀alkoxy group is to be understood as being a straight-chain or branched alkoxy group, e.g. methoxy, ethoxy, n-propoxy, isopropoxy, n-butoxy, sec-butoxy, tert-butoxy, pentyloxy, isopentyloxy, tert-pentyloxy, heptyloxy, octyloxy, isooctyloxy, nonyloxy, decyloxy and the like.

A C₁-C₆alkylcarbonyl group is to be understood as being a straight-chain or branched carbonyl group with an alkyl radical having from one to six carbon atoms, e.g. acetyl, propionyl, butyryl, isobutyryl, valeryl, isovaleryl, pivaloyl and the like.

Suitable hydroxy-substituted C₁-C₂₀alkyl groups are e.g. hydroxymethyl, hydroxyethyl, hydroxypropyl, hydroxybutyl, hydroxypentyl, hydroxyhexyl, hydroxyheptyl, hydroxyoctyl, hydroxynonyl, hydroxydecyl and the like.

Compounds of very particular interest are those of formula (I) wherein
R₁ is C₁-C₁₆alkyl; and
R₂, R₃, R₄ and R₅ are hydrogen.

Preferred arylsulfatase-inhibiting substance are selected from hydroxydiphenyl ethers are formula (II) having 4-phenoxyphenol as the underlying framework 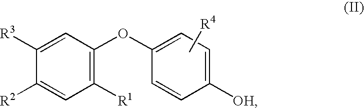
wherein
R₁ and R₂ are each independently of the other a hydrogen atom, a hydroxy group or a C₁-C₂₀alkyl, C₅-C₇cycloalkyl, C₁-C₆alkylcarbonyl, C₁-C₂₀alkoxy, phenyl or phenyl-C₁-C₃alkyl group,
R₃ is a hydrogen atom or a C₁-C₂₀alkyl or C₁-C₂₀alkoxy group and R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
   hydroxydiphenyl ethers of general formula (III) having 3-phenoxyphenol as the underlying framework 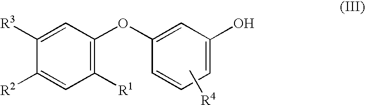
   wherein
R₂ is a hydrogen atom or a C₁-C₂₀alkyl, hydroxy-substituted C₁-C₂₀alkyl or C₁-C₆-alkylcarbonyl group,
R₁ and R₃ are each independently of the other a hydrogen atom, a C₁-C₆alkylcarbonyl group or a C₁-C₂₀alkyl group and
R₄ is a hydrogen atom or a C₁-C₂₀alkyl, hydroxy-substituted C₁-C₂₀alkyl, C₁-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
   and hydroxydiphenyl ethers of general formula (IV) having 2-phenoxyphenol as the underlying framework 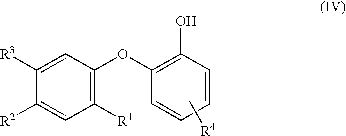
   wherein
R₁ is a hydrogen atom or a C₁-C₆alkylcarbonyl or C₁-C₂₀alkyl group,
R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group and
R₂ and R₃ are each independently of the other a hydrogen atom or a C₁-C₆alkylcarbonyl or C₁-C₂₀alkyl group, in a cosmetic deodorant or antiperspirant composition for reducing body odour caused by the hydrolytic decomposition of steroid esters.

Compounds of very most particular interest are those of formula 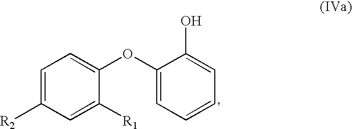
R₁ and R₂ independently from each other are hydrogen; C₁-C₂₀alkyl; or hydroxy-substituted C₁-C₂₀ alkyl; wherein at least one of the substituents R₁ and R₂ is not hydrogen.

Furthermore, compounds of formula 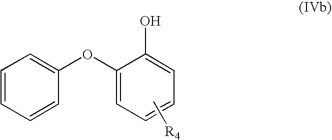
are of particular interest, wherein
R₄ is carboxy; carboxy-C₁-C₃alkyl; C₁-C₆alkylcarbonyl; or C₁-C₃alkylcarbonyl-C₁-C₃alkyl.

Furthermore, compounds of formula 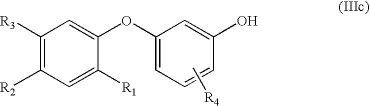
are of particular interest, wherein
R₁, R₂ and R₃ independently from each other are hydrogen; hydroxy; C₁-C₂₀alkyl; or hydroxy-substituted C₁-C₂₀alkyl; and
R₄ is C₁-C₂₀alkyl; hydroxysubstituted C₁-C₂₀alkyl; phenyl; phenyl-C₁-C₂0alkyl; or C₁-C₆alkylcarbonyl;
   and most preferably compounds of formula (IIIc), wherein
R₁, R₁ and R₃ are hydrogen; or C₁-C₂₀alkyl.

Most preferably are compounds of formula (IIIc), wherein
R₁, R₂ and R₃ are hydrogen; and
R₄ is C₁-C₂₀alkyl; phenyl-C₁-C₅alkyl; or C₁-C₆alkylcarbonyl.

Furthermore, compounds of formula 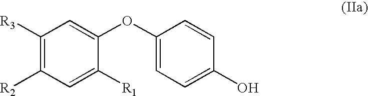
are preferably used, wherein
R₁, R₂ and R₃ independentiy from each other are hydrogen; C₁-C₂₀alkyl; hydroxy-substituted C₁-C₂₀alkyl; cyclo-C₅-C₇alykl; phenyl-C₁-C₃alkyl, wherein at least one of the radicals R₁, R₁, or R₃ is not hydrogen.

Most preferred are compounds of formula (IIa), wherein
R₁and R₂ independently from each other are C₁-C₂₀alkyl; most preferably C₁-C₅alkyl; and
R₂ is hydrogen.

Preferred hydroxydiphenyl ethers are those of general formula (II) wherein
R₁ and R₂ are each independently of the other a hydrogen atom or a C₁-C₂₀alkyl, C₁-C₆alkylcarbonyl or C₁-C₂₀alkoxy group,
R₃ is a hydrogen atom or a C₁-C₂₀alkyl or C₁-C₂₀alkoxy group and
R₄ is a hydrogen atom or a C₁-C₂₀alkyl, hydroxy-substituted C₁-C₂₀alkyl, C₁-C₆alkylcarbonyl, hydroxy, formyl, acetonyl, allyl, carboxymethyl or carboxyallyl group,
   hydroxydiphenyl ethers of general formula (III) wherein
R₂ is a hydrogen atom or a C₁-C₂₀alkyl, hydroxy-substituted C₁-C₂₀alkyl or C₁-C₆- alkylcarbonyl group,
R₁ and R₃ are each independently of the other a hydrogen atom, a C₁-C₆alkylcarbonyl group or a C₁-C₂₀ alkyl group and
R₄ is a hydrogen atom or a C₁-C₂₀alkyl, hydroxy-substituted C₁-C₂₀alkyl, hydroxy, formyl, acetonyl, allyl, carboxymethyl, carboxyallyl or C₁-C₆alkylcarbonyl group,
   and hydroxydiphenyl ethers of general formula (IV) wherein
R₁ ,is a hydrogen atom or a C₁-C₆alkylcarbonyl or C₁-C₂₀alkyl group,
R₄ is a hydrogen atom or a C₁-C₂₀alkyl, hydroxy-substituted C₁-C₂₀alkyl, hydroxy, formyl, acetonyl, allyl, carboxymethyl, C₁-C₆alkylcarbonyl or carboxyallyl group and
R₂ and R₃ are each independently of the other a hydrogen atom or a C₁-C₆alkylcarbonyl or C₁-C₂₀alkyl group.

Further hydroxydiphenylether compound, which can be used in the present invention are listed in the Table 1 below:

TABLE 1

| | | | |
|---|---|---|---|
| HD-01 | 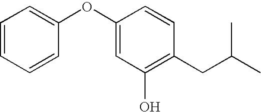 | HD-02 | 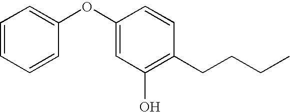 |
| HD-03 | 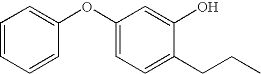 | HD-04 | 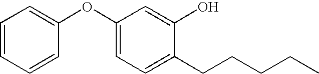 |
| HD-05 | 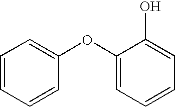 | HD-06 | 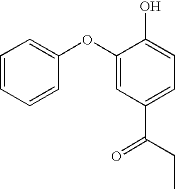 |
| HD-07 | 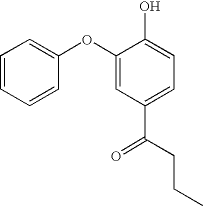 | HD-08 | 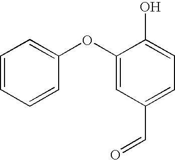 |
| HD-09 | 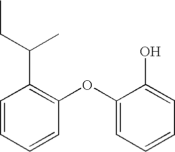 | HD-10 | 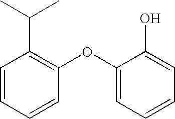 |
| HD-11 | 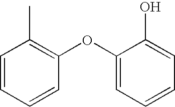 | HD-12 | 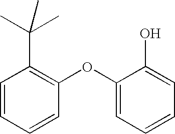 |
| HD-13 | 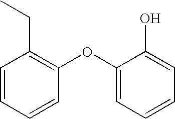 | HD-14 | 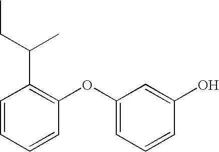 |
| HD-15 | 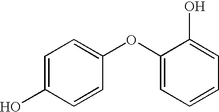 | HD-16 | 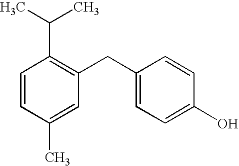 |
| HD-17 | 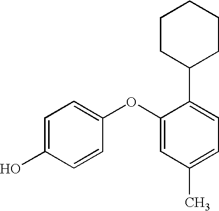 | HD-18 | 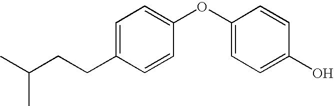 |
| HD-19 | 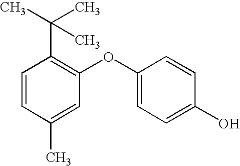 | HD-20 | 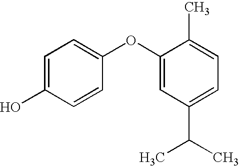 |
| HD-21 | 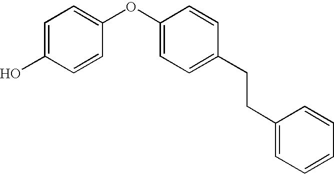 | HD-22 | 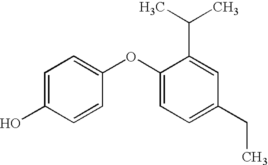 |
| HD-23 | 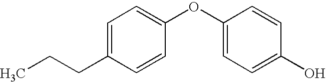 | HD-24 | 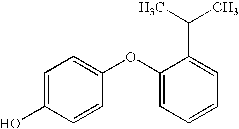 |
| HD-25 | 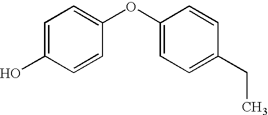 | HD-26 | 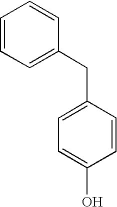 |
| HD-27 | 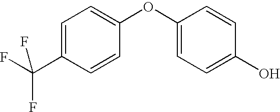 | HD-28 | 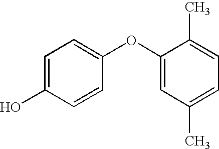 |
| HD-29 | 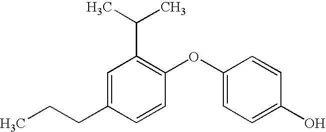 | HD-30 | 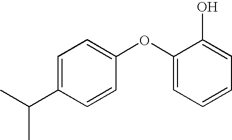 |
| HD-31 | 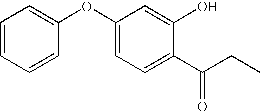 | HD-32 | 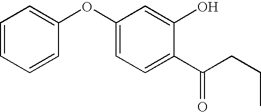 |
| HD-33 | 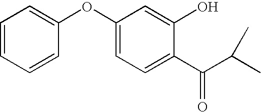 | HD-34 | 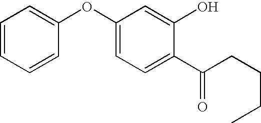 |
| HD-35 | 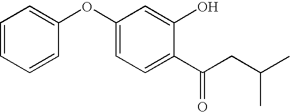 | HD-36 | 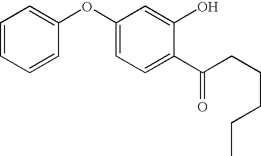 |
| HD-37 | 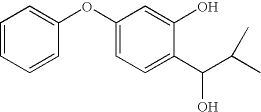 | HD-38 | 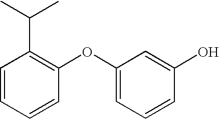 |
| HD-39 | 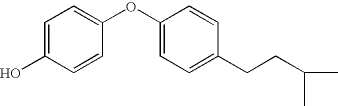 | HD-40 | 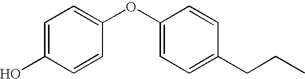 |
| HD-41 | 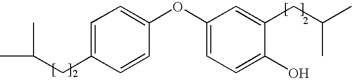 | HD-42 | 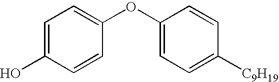 |
| HD-43 | 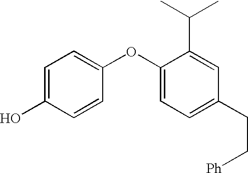 | HD-44 | 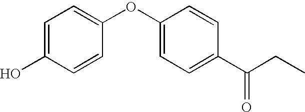 |
| HD-45 | 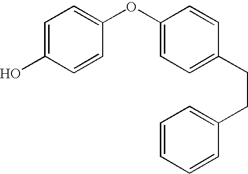 | HD-46 | 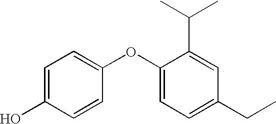 |
| HD-47 | 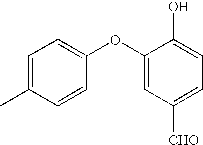 | HD-48 | 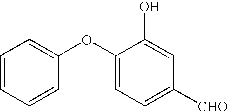 |
| HD-49 | 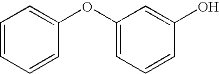 | HD-50 | 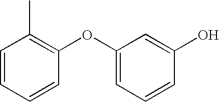 |
| HD-51 | 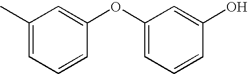 | HD-52 | 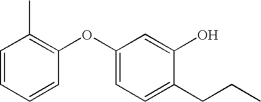 |
| HD-53 | 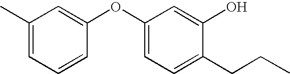 | HD-54 | 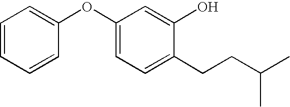 |
| HD-55 | 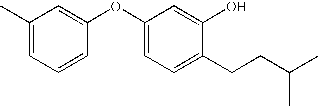 | HD-56 | 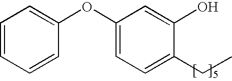 |
| HD-57 | 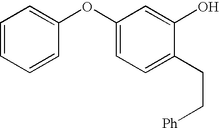 | HD-58 | 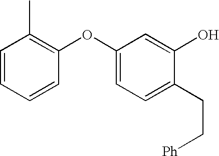 |
| HD-59 | 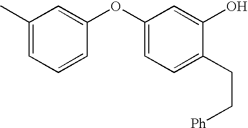 | HD-60 | 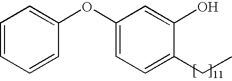 |
| HD-61 | 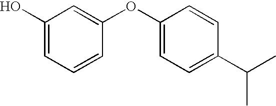 | HD-62 | 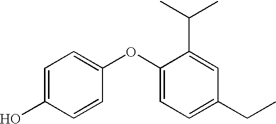 |
| HD-63 | 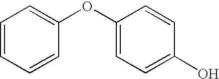 | HD-64 | 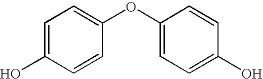 |
| HD-65 | 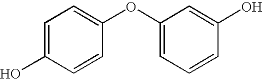 | HD-66 | 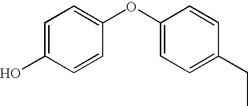 |

The present invention relates also to a method of reducing body odour by means of the inhibition of arylsulfatase on the skin, wherein a cosmetic deodorant or antiperspirant composition comprising at least one arylsulfatase-inhibiting substance selected from hydroxydiphenyl ethers of general formula 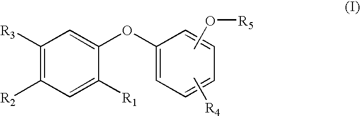
wherein
R₁, R₂ and R₃ independently from each other are hydrogen; hydroxy; C₁-C₂₀alkyl; hydroxy-substituted C₁C₂₀alkyl; C₁-C₂₀cycloalkyl; C₁-C₂₀alkoxy; C₁-C₆alkylcarbonyl; phenyl; or phenyl-C₁-C₃alkyl;
R₄ hydrogen, C₁-C₂₀ alkyl; hydroxy-substitute C₁-C₂₀alkyl; C₅C₇-cycloalkyl; hydroxy; formyl; acetonyl; allyl; carboxy; carboxy-C₁-C₃alkyl; carboxyallyl; C₂-C₂₀alkenyl; C₁-C₆-alkyl-carbonyl; C₁-C₃alkylcarbonyl-C₁-C₃alkyl; phenyl; or phenyl-C₁-C₃alkyl; and
R₅ is hydrogen; C₁-C₂₀alkoxy; or C₁-C₆alkylcarbonyl.

Preferably the cosmetic deodorant or antiperspirant composition comprises at least one arylsulfatase-inhibiting substance selected from hydroxydiphenyl ethers of general formula 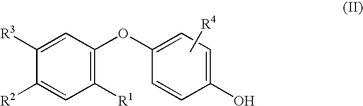
wherein
R₁ and R₂ are each independently of the other a hydrogen atom, a hydroxy group or a C₁-C₂₀alkyl, C₁-C₇cycloalkyl, C₁-C₆alkylcarbonyl, C₁-C₂₀alkoxy, phenyl or phenyl-C₁-C₃alkyl group,
R₃ is a hydrogen atom or a C₁-C₂₀alkyl or C₁-C₂₀alkoxy group and
R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
   hydroxydiphenyl ethers of general formula 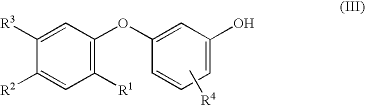
   wherein
R₂ is a hydrogen atom or a C₁-C₂₀alkyl, hydroxy-substituted C₁-C₂₀alkyl or C₁-C₆-alkylcarbonyl group,
R₁ and R₃ are each independently of the other a hydrogen atom, a C₁-C₆alkylcarbonyl group or a C₁-C₂₀alkyl group and
R₄ is a hydrogen atom or a C₁-C₂₀alkyl, hydroxy-substituted C₁-C₂₀alkyl, C₁-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
   and hydroxydiphenyl ethers of general formula 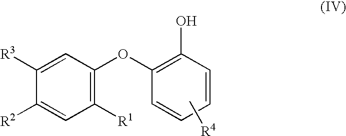
   wherein
R₁ is a hydrogen atom or a C₁-C₆alkylcarbonyl or C₁-C₂₀alkyl group,
R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group and
R₂ and R₃ are each independently of the other a hydrogen atom or a C₁-C₆alkylcarbonyl or C₁-C₂₀alkyl group,
   is applied to the skin, especially to the skin of the armpits.

Especially suitable hydroxydiphenyl ethers having 4-phenoxyphenol as the underlying framework are: the compounds 4-(2,5-dimethylphenoxy)phenol, 4-(3-isopropyl-6-methyl-phenoxy)phenol, 4-(2-tert-butyl-5-methylphenoxy)phenol, 4-(2-cyclohexyl-5-methyl-phenoxy)phenol, 4-(2-isopropyl-5-methylphenoxy)phenol and 4-(3-isopropyl-5-methyl-phenoxy)phenol,
having 3-phenoxyphenol as the underlying framework: the compounds 3-(2-isopropyl-phenoxy)phenol, 3-(2-sec-butylphenoxy)phenol, 3-phenoxy-6-propylphenol, 3-phenoxy-6-(2-methylpropyl)phenol, 3-phenoxy-6-butylphenol, 3-phenoxy-6-pentylphenol, 3-phenoxy-6-hexylphenol, 3-phenoxy-6-(3-methylbutanoyl)phenol and 3-phenoxy-6-hexanoylphenol,
and having 2-phenoxyphenol as the underlying framework: the compounds 2-(2-ethyl-phenoxy)phenol, 2-(2-isopropylphenoxy)phenol, 2-(2-sec-butylphenoxy)phenol and 2-(2-tert-butylphenoxy)phenol.

The hydroxydiphenyl ethers used according to the invention are employed in amounts of from 0.001 to 2% by weight, preferably from 0.005 to 1.0% by weight, and especially from 0.01 to 0.5% by weight, in each case based on the weight of the total deodorant or antiperspirant composition.

The cosmetic deodorant or antiperspirant compositions comprising the arylsulfatase-inhibitors used according to the invention can be in the form of powders, in stick form, in the form of an aerosol spray, a pump-action spray, a liquid or gel-form roll-on form of application, a cream, a gel and in the form of an impregnated flexible substrate. Deodorant or antiperspirant sticks can be in gelled form, based on anhydrous wax and based on W/O emulsions and O/W emulsions. Gel sticks can be prepared on the basis of fatty acid soaps, dibenylidene sorbitol, N-acylamino acid amides, 12-hydroxystearic acid and other gel formers.

An aerosol spray, pump-action spray, a roll-on form of application or cream can be in the form of a water-in-oil emulsion, oil-in-water emulsion, silicone oil-in-water emulsion, water-in-oil microemulsion, oil-in-water microemulsion, silicone oil-in-water microemulsion, non-aqueous suspension, alcoholic or hydroalcoholic solution, aqueous gel or an oil. All the mentioned compositions can be thickened, for example on the basis of fatty acid soaps, dibenzylidene sorbitol, N-acylamino acid amides, 12-hydroxystearic acid, polyacrylates of the carbomer and carbopol type, polyacrylamides and polysaccharides, which may be chemically and/or physically modified.

The emulsions and microemulsions can be transparent, translucent or opaque.

The cosmetic deodorant or antiperspirant compositions comprising the arylsulfatase-inhibitors used according to the invention may also comprise fatty substances. Fatty substances are to be understood as being fatty acids, fatty alcohols, natural and synthetic cosmetic oil components and also natural and synthetic waxes, which, based on a temperature of 25° C., can be in solid form or in liquid form or in aqueous or oily dispersion.

Fatty acids are able to form gels and can thus be used in the preparation of solidified stick formulations. As fatty acids there may be used linear and/or branched, saturated and/or unsaturated C₈₋₃₀fatty acids. C₁₀₋₂₂Fatty acids are preferred. Examples are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical mixtures thereof. The use of stearic acid is especially preferred. The fatty acids used may carry one or more hydroxy groups.

Preferred examples thereof are α-hydroxy-C₈-C₁₈carboxylic acids and also 12-hydroxystearic acid.

Fatty alcohols and other fatty substances that are solid at 25° C. are likewise preferably used in the preparation of stick formulations. Fatty alcohols that may be used include saturated or mono- or poly-unsaturated, branched or unbranched fatty alcohols having from 6 to 30, preferably from 10 to 22 and especially from 12 to 22 carbon atoms. Suitable for use according to the invention are e.g. octanol (capryl alcohol), octenol, octadienol, decanol (capric alcohol), decenol, decadienol, dodecanol (lauryl alcohol), dodecenol, dodecadienol, oleyl alcohol, erucyl alcohol, ricinoleyl alcohol, stearyl alcohol, isostearyl alcohol, cetyl alcohol, myristyl alcohol, arachidyl alcohol, linoleyl alcohol, linolenyl alcohol and behenyl alcohol and also the Guerbet alcohols thereof.

Waxes are frequently used for stick formulations. As natural or synthetic waxes it is possible to use according to the invention solid paraffins or isoparaffins, vegetable waxes, such as candelilla wax, carnauba wax, esparto wax, japan wax, cork wax, sugarcane wax, ouricury wax, montan wax, sunflower wax, fruit waxes and animal waxes, e.g. beeswax and other insect waxes, spermaceti, shellac wax, wool wax and preen gland oil, also mineral waxes, e.g. ceresin and ozocerite, or the petrochemical waxes, e.g. petrolatum, paraffin waxes, microwaxes of polyethylene or polypropylene, and polyethylene glycol waxes. It may be advantageous to use hydrogenated or hardened waxes. Also suitable for use are chemically modified waxes, especially the hard waxes, e.g. montan ester waxes, sasol waxes and hydrogenated jojoba waxes.

Also suitable are the triglycerides of saturated and optionally hydroxylated C₁₆₋₃₀fatty acids, e.g. hardened triglyceride fats (hydrogenated palm oil, hydrogenated coconut oil, hydrogenated castor oil), glyceryl tribehenate or glyceryl tri-12-hydroxystearate, also synthetic full esters of fatty acids and glycols (e.g. Syncrowachs®) or polyols having from 2 to 6 carbon atoms, fatty acid monoalkanolamides having a C₁₂₋₂₂acyl radical and a C₂₋₄alkanol radical, esters of saturated and/or unsaturated, branched and/or unbranched alkanecarboxylic acids having a chain length of from 1 to 80 carbon atoms and saturated and/or unsaturated, branched and/or unbranched alcohols having a chain length of from 1 to 80 carbon atoms, including e.g. synthetic fatty acid fatty alcohol esters such as stearyl palmitate or cetyl palmitate, esters of aromatic carboxylic acids, dicarboxylic acids and hydroxycarboxylic acids (e.g. 12-hydroxystearic acid) and saturated and/or unsaturated, branched and/or unbranched alcohols having a chain length of from 1 to 80 carbon atoms, lactides of long-chain hydroxycarboxylic acids and full esters of fatty alcohols and di- and tri-carboxylic acids, e.g. dicetyl succinate or dicetyl/stearyl adipate, and also mixtures of such substances, provided that the individual wax components or their mixtures are solid at 25° C.

It is especially preferred to select the wax components from the group of esters of saturated, unbranched alkanecarboxylic acids having a chain length of from 14 to 44 carbon atoms and saturated, unbranched alcohols having a chain length of from 14 to 44 carbon atoms, provided that the wax component or the totality of wax components is solid at 25° C. It is especially advantageous to select the wax components from the group of C₁₆₋₃₆alkyl stearates, C₂₀₋₄₀alkyl isostearates, C₂₀₋₄₀dialkyl esters of dimeric acids, C₁₈₋₃₈alkylhydroxystearoyl stearates and C₂₀₋₄₀alkyl erucates; also suitable for use are C₃₀₋₅₀alkyl beeswax and cetearyl behenate. Silicone waxes, for example stearyl trimethylsilane/stearyl alcohol, may also be advantageous. Especially preferred wax components are the esters of saturated, monohydric C₂₀-C₆₀alcohols and saturated C₈-C₃₀monocarboxylic acids, especially a C₂₀-C₄₀alkyl stearate, which is obtainable under the name Kesterwachs® K82H from Koster Keunen Inc. The wax or the wax components should be solid at 25° C., but should melt in the range of from 35 to 95° C., preference being given to a range of from 45 to 85° C.

Natural, chemically modified and synthetic waxes can be used alone or in combination.

The gel formers and solidifying components are present in an amount of from 0.1 to 50% by weight, based on the total composition used according to the invention, preferably from 1 to 40% by weight and especially from 5 to 30% by weight.

The compositions used according to the invention can also comprise at least one non-polar or polar liquid oil, which may be natural or synthetic.

The polar oil component can be selected from vegetable oils, e.g. sunflower oil, olive oil, soybean oil, rape oil, almond oil, jojoba oil and the liquid components of coconut oil, and also from synthetic triglyceride oils, from ester oils, that is to say esters of C₆₋₃₀fatty acids with C₂₋₃₀fatty alcohols, from dicarboxylic acid esters such as di-n-butyl adipate, di(2-ethyl-hexyl) adipate and di(2-ethylhexyl) succinate, and also diol esters such as ethylene glycol dioleate and propylene glycol di(2-ethylhexanoate), from symmetric, asymmetric or cyclic esters of carbonic acid with fatty alcohols, described for example in DE-OS 197 56 454, glycerol carbonate or dicaprylyl carbonate (Cetiol® CC), from mono-, di- and tri-fatty acid esters of saturated and/or unsaturated linear and/or branched fatty acids with glycerol, from branched alkanols, e.g. Guerbet alcohols having a single branch at carbon atom 2, such as 2-hexyl decanol, 2-octyl dodecanol, isotridecanol and isohexadecanol, from alkanediols, e.g. the vicinal diols obtainable from epoxyalkanes having from 12 to 24 carbon atoms by ring-opening with water, from ether alcohols, e.g. the monoalkyl ethers of glycerol, of ethylene glycol, of 1,2-propylene glycol or of 1,2-butanediol, from dialkyl ethers each having from 12 to 24 carbon atoms, e.g. alkyl methyl ethers or di-n-alkyl ethers each having a total of from 12 to 24 carbon atoms, especially di-n-octyl ether (Cetiol®OE from Cognis), and also from addition products of ethylene oxide and/or propylene oxide with mono- or poly-valent C₃₋₂₀alkanols such as butanol and glycerol, e.g. PPG-3 myristyl ether (Witconol® APM), PPG-14 butyl ether (Ucon Fluid® AP), PPG-15 stearyl ether (Arlamol® E), PPG-9 butyl ether (Breox® B25) and PPG-10 butanediol (Macol® 57).

The non-polar oil component can be selected from liquid paraffin oils, isoparaffin oils, e.g. isohexadecane and isoeicosane, from synthetic hydrocarbons, e.g. 1,3-di(2-ethyl-hexyl)cyclohexane (Cetiol® S), and from volatile and non-volatile silicone oils, which may be cyclic, e.g. decamethylcyclopentasiloxane and dodecamethylcyclohexasiloxane, or linear, e.g. linear dimethylpolysiloxane, commercially available e.g. under the name Dow Corning® 190, 200, 244, 245, 344 or 345 and Baysilon® 350 M.

The compositions used according to the invention may also comprise at least one water-soluble alcohol. Water-solubility is to be understood according to the invention as meaning that at least 5% by weight of the alcohol dissolve at 20° C. to form a clear solution or—in the case of long-chain or polymeric alcohols—can be dissolved by heating the solution to from 50° C. to 60° C. Depending upon the form of application, monohydric alcohols, e.g. ethanol, propanol or isopropanol, are suitable. Also suitable are water-soluble polyols. These include water-soluble diols, triols and higher valency alcohols, and also polyethylene glycols. Suitable diols include C₂-C₁₂diols, especially 1,2-propylene glycol, butylene glycols, e.g. 1,2-butylene glycol, 1,3-butylene glycol and 1,4-butylene glycol, hexanediols, e.g. 1,6-hexanediol. Also especially suitable are glycerol and more especially diglycerol and triglycerol, 1,2,6-hexanetriol and the polyethylene glycols (PEG) PEG-400, PEG-600, PEG-1000, PEG-1550, PEG-3000 and PEG4000.

The amount of alcohol or alcohol mixture in the compositions used according to the invention is from 1 to 50% by weight and preferably from 5 to 40% by weight, based on the total composition. According to the invention either one alcohol or a mixture of a plurality of alcohols can be used.

The compositions used according to the invention can be substantially non-aqueous, that is to say they can contain a maximum of 5% by weight, preferably a maximum of 1% by weight, water. In water-containing forms of application, the water content is from 5 to 98% by weight, preferably from 10 to 90% by weight and especially from 15 to 85% by weight, based on the total composition.

The compositions used according to the invention can also comprise at least one hydrophilically modified silicone. They enable highly transparent compositions to be formulated, they reduce stickiness and they leave behind a fresh feeling on the skin. Hydrophilically modified silicones are to be understood according to the invention as being polyorganosiloxanes having hydrophilic substituents which affect the water-solubility of the silicones. According to the invention, water-solubility is to be understood as meaning that at least 2% by weight of the silicone that has been modified with hydrophilic groups dissolve in water at 20° C. Examples of suitable hydrophilic substituents are hydroxy, polyethylene glycol or polyethylene glycol/polypropylene glycol side chains or end groups and also ethoxylated ester side chains or end groups. Especially suitable in accordance with the invention are hydrophilically modified silicone copolyols, especially dimethicone copolyols, which are commercially available, for example, from Wacker-Chemie under the name Belsil® DMC 6031, Belsil® DMC 6032, Belsil® DMC 6038 or Belsil® DMC 3071 VP and from Dow Coming under the name DC 2501.

The compositions used according to the invention can also comprise at least one water-soluble surfactant. Suitable water-soluble surfactants are in principle any surfactants that are 1% by weight soluble in the system at 20° C. and at least 1% by weight soluble in water at 20° C. Although the structure and ionicity are unimportant per se, it appears that non-ionic surfactants are especially suitable, more especially the addition products of ethylene oxide with fatty molecules having at least one group capable of alkoxylation that are solid at normal temperature (20° C.). Examples of such suitable surfactants are the addition products of from 10 to 40 mol of ethylene oxide with linear fatty alcohols having from 16 to 22 carbon atoms, with fatty acids having from 12 to 22 carbon atoms, with fatty acid alkanolamides, with fatty acid monoglycerides, with sorbitan fatty acid monoesters, with fatty acid alkanolamides, with fatty acid glycerides, e.g. hardened castor oil, with methylglucoside monofatty acid esters, and mixtures thereof

The compositions used according to the invention comprise in a preferred embodiment at least one antiperspirant active ingredient. Suitable antiperspirant active ingredients are water-soluble astringent metal salts, especially inorganic and organic salts of aluminium, zirconium and zinc, and any mixtures of such salts. According to the invention, water-solubility is to be understood as being a solubility of at least 5 g of active ingredient per 100 g of solution at 20° C. Suitable for use according to the invention are, for example, alum(KAI(SO₄)₂.12H₂O), aluminium sulfate, aluminium lactate, sodium aluminium chlorohydroxylactate, aluminium chlorohydroxyallantoinate, aluminium chlorohydrate, aluminium sulfocarbolate, aluminium zirconium chlorohydrate, zinc chloride, zinc sulfo-carbolate, zinc sulfate, zirconium chlorohydrate and aluminium zirconium chlorohydrate glycine complexes. The compositions preferably comprise an astringent aluminium salt, especially aluminium chlorohydrate, and/or an aluminium zirconium compound. In the case of aqueous forms of application the antiperspirant active ingredients are used in the form of aqueous solutions. In non-aqueous compositions, the antiperspirant active ingredients are used in solid form. They are present in the compositions used according to the invention in an amount of from 1 to 40% by weight, preferably from 5 to 30% by weight and especially from 8 to 25% by weight (based on the amount of active ingredient in the total composition). Aluminium chlorohydrates are available in pulverulent form, e.g. Micro Dry® Ultrafine from Reheis, in activated form, e.g. Reach® 501 from Reheis, and in the form of aqueous solutions, e.g. Locron® L from Clariant or Chlorhydrol® from Reheis. An aluminium sesquichlorohydrate is available from Reheis under the name Reach® 301. The use of aluminium zirconium tetrachlorohydrex-glycine complexes, which are commercially available e.g. from Reheis under the name Rezal® 36G, is also advantageous according to the invenbon. Activated aluminium zirconium polymers having a low molecular weight are also used. Also suitable as deodorants according to the invention are fragrances, antimicrobial, anti-bacterial or microorganism-inhibiting substances, enzyme-inhibiting substances, anti-oxidants and odour absorbers.

Suitable antimicrobial, antibacterial or microorganism-inhibiting substances are especially C₁-C₄alkanols, C₂-C₄alkanediols, organohalogen compounds and organohalides, quaternary ammonium compounds and a range of plant extracts and zinc compounds. Preference is given to halogenated phenol derivatives, e.g. hexachlorophene or Irgasan DP 300 (triclosan, 2,4,4′-trichloro-2′-hydroxydiphenyl ether), 3,4,4′-trichlorocarbonilide, chlorhexidine (1,1′-hexamethylene-bis[5-(4-chlorophenyl)]biguanide), chlorhexidine gluconate, benzalkonium halides and cetylpyridinium chloride. Also suitable for use are sodium hydrogen carbonate, sodium phenol sulfonate and zinc phenol sulfonate and e.g. the constituents of linden blossom oil. Antimicrobial substances that have a weaker action but exhibit a specific action against the gram-positive microorganisms responsible for the decomposition of sweat can also be used as deodorant active ingredients. Such substances include many ethereal oils, e.g. oil of cloves (eugenol), mint oil (menthol) or thyme oil (thymol) as well as terpene alcohols, e.g. farnesol. It is also possible to use aromatic alcohols, e.g. benzyl alcohol, 2-phenylethanol or 2-phenoxyethanol, as deodorant active ingredients. Further antibacterially active deodorants are lantibiotics, glycoglycerolipids, sphingolipids (ceramides), sterols and other active ingredients that inhibit the adhesion of bacteria to the skin, e.g. glycosidases, lipases, proteases, carbohydrates, di- and oligo-saccharide fatty acid esters and also alkylated mono- and oligo-saccharides. Likewise suitable are long-chain diols, e.g. 1,2-alkane(C₈-C₁₈)diols, glycerol mono(C₆-C₁₆)-alkyl ethers and glycerol mono(C₈-C₁₈)fatty acid esters, which are very well tolerated by the skin and mucosa and are active against corynebacteria.

Enzyme-inhibiting substances having a deodorising action are primarily those which inhibit the ester-cleaving enzymes and in that way counteract the decomposition of sweat. Especially suitable for this purpose are zinc salts, plant extracts, e.g. citrus seed extracts, and the esters of aliphatic C₂-C₆carboxylic acids or hydroxycarboxylic acids and C₂-C₆alcohols or polyols, e.g. triethyl citrate, propylene glycol lactate or glycerol triacetate (triacetin).

Anti-oxidative substances are able to counteract the oxidative decomposition of the constituents of sweat and in that way inhibit the development of odour. Suitable anti-oxidants are tocopherols and derivatives thereof, especially tocopheryl acetate, retinoids, especially retinol and retinyl palmitate, carotinoids, carotins (e.g. α-carotin, β-carotin, lycopene) and derivatives thereof, liponic acid and derivatives thereof (e.g. dihydrolipoic acid), thio compounds, e.g. thioglycerol, thiosorbitol, thioglycolic acid, thioredoxin, glutathione, cysteine, cystine, cystamine and esters thereof and also salts thereof, dilauryl thiodipropionate, distearyl thiodipropionate, thiodipropionic acid and derivatives thereof, and also sulfoximine compounds in very small tolerable concentrations (e.g. pmol/kg to μmol/kg), also metal-chelating agents (e.g. α-hydroxyfatty acids, EDTA, EGTA, phytic acid, lactoferrin), humic acids, bile acid, bile extracts, gallic acid esters (e.g. propyl, octyl and dodecyl gallate), flavonoids, catechols, bilirubin, biliverdin and derivatives thereof, folic acid and derivatives thereof, hydroquinone and derivatives thereof (e.g. arbutin), ubiquinone and ubiquinol and derivatives thereof, isoascorbic acid and derivatives thereof, rutin, rutinic acid and derivatives thereof, disodium rutinyl disulfate, cinnamic acid and derivatives thereof (e.g. ferulic acid, ethyl ferulate, caffeic acid), kojic acid, chitosan glycolate and salicylate, butyl hydroxytoluene, butyl hydroxyanisole, nordihydroguaiaretic acid, trihydroxybutyrophenone, uric acid and derivatives thereof, mannose and derivatives thereof, selenium and selenium derivatives (e.g. selenium methionine), stilbene and stilbene derivatives (e.g. stilbene oxide, trans-stilbene oxide). According to the invention it is possible to use suitable derivatives (salts, esters, sugars, nucleotides, nucleosides, peptides and lipids) as well as mixtures of the mentioned active ingredients or plant extracts (e.g. tea tree oil, rosemary extract and rosemarinic acid) containing such antioxidants.

As lipophilic, oil-soluble antioxidants from that group preference is given to gallic acid esters, flavonoids and carotinoids and also butylhydroxytoluene/anisole. As water-soluble antioxidants preference is given to tannins, especially those of vegetable origin.

The total amount of antioxidants in the compositions used according to the invention is from 0.001 to 10% by weight, preferably from 0.05 to 5% by weight and especially from 0.05 to 2% by weight, based on the total composition.

The following substances can be used as odour absorbers: zinc ricinoleate, cyclodextrin and derivatives thereof, hydroxypropyl-β-cyclodextrin, also oxides such as magnesium oxide or zinc oxide, the oxides not being compatible with aluminium chlorohydrate, also starch and starch derivatives, silicic acids, which may or may not be modified, zeolites, talcum and also synthetic polymers, e.g. nylon. The deodorising action may also be supported by complex-forming substances because they form a stable complex with the oxidatively catalytically active heavy metal ions (e.g. iron or copper). Suitable complex formers are e.g. the salts of ethylenediaminetetraacetic acid or nitrilotriacetic acid and also the salts of 1 -hydroxyethane-1,1-diphosphonic acid.

Suitable fragrances and perfume oils are, for example, mixtures of natural and/or synthetic aromatic substances. Natural aromatic substances are extracts from blossom (lilies, lavender, roses, jasmine, neroli, ylang-ylang), from stems and leaves (geranium, patchouli, petitgrain), from fruit (aniseed, coriander, carraway, juniper), from fruit peel (bergamot, lemons, oranges), from roots (mace, angelica, celery, cardamom, costus, iris, calamus), from wood (pinewood, sandalwood, guaiacum wood, cedarwood, rosewood), from herbs and grasses (tarragon, lemon grass, sage, thyme), from needles and twigs (spruce, pine, Scots pine, mountain pine) and from resins and balsams (galbanum, elemi, benzoin, myrrh, olibanum, opoponax). Animal raw materials also come into consideration, for example civet and castoreum. Typical synthetic aromatic substances are products of the ester, ether, aldehyde, ketone, alcohol or hydrocarbon type. Aromatic substance compounds of the ester type are, for example, benzyl acetate, phenoxyethyl isobutyrate, p-tert-butylcyclohexyl acetate, linalyl acetate, dimethyl-benzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; thealdehydes include, for example, the linear alkanals having from 8 to 18 carbon atoms, citral, citronellal, citronellyl oxyacetaldehyde, cyclamen aldehyde, hydroxycitronellal, lilial and bourgeonal; the ketones include, for example, the ionones, α-isomethylionone and methyl cedryl ketone; the alcohols include anethol, citronellol, eugenol, isoeugenol, geraniol, linalool, phenyl ethyl alcohol and terpineol; and the hydrocarbons include mainly the terpenes and balsams. It is preferable, however, to use mixtures of different aromatic substances that together produce an attractive scent.

Ethereal oils of relatively low volatility, which are chiefly used as aroma components, are also suitable as perfume oils, e.g. sage oil, camomile oil, clove oil, melissa oil, mint oil, cinnamon leaf oil, linden blossom oil, juniper berry oil, vetiver oil, olibanum oil, galbanum oil, labdanum oil and lavandin oil.

Preference is given to the use of bergamot oil, dihydromyrcenol, lilial, lyral, citronellol, phenyl ethyl alcohol, α-hexylcinnamaldehyde, geraniol, benzyl acetone, cyclamen aldehyde, linalool, boisambrene forte, ambroxan, indole, hedione, sandelice, lemon oil, tangerine oil, orange oil, allyl amyl glycolate, cyclovertal, lavandin oil, muscatel sage oil, β-damascone, bourbon geranium oil, cyclohexyl salicylate, vertofix coeur, iso-E-Super, Fixolide®NP, evernyl, iraldein gamma, phenylacetic acid, geranyl acetate, benyl acetate, rose oxide, romillat, irotyl and floramat alone or in admixture with one another.

According to the invention, the perfume oil and/or ethereal oil is present in amounts of from 0.01 to 2% by weight, preferably from 0.1 to 1% by weight, in each case based on the weight of the total composition used according to the invention.

Liquid and gel-form forms of application may comprise thickeners, e.g. cellulose ethers, such as hydroxypropylcellulose, hydroxyethylcellulose and methylhydroxypropylcellulose, thickening polymers based on polyacrylates, which may if desired be crosslinked, e.g. the carbopol types or Pemulen® products, or based on polyacrylamides or sulfonic-acid-group-containing polyacrylates, e.g. Sepigel® 305 or Simulge® EG, also inorganic thickeners, e.g. bentonites and hectorites (Laponite®).

The compositions used according to the invention may comprise further cosmetically and dermatologically effective substances, for example antiinflammatory substances, solids, selected from silicic acids, e.g. Aerosil® types, silica gels, silicon dioxide, clays, e.g. bentonites or kaolin, magnesium aluminium silicates, e.g. talcum, boron nitride, titanium dioxide, which may be coated if desired, unmodified and modified starches and starch derivatives, cellulose powders and polymer powders, also plant extracts, protein hydrolysates and vitamins.

The cosmetic deodorant or antiperspirant compositions comprising the arylsulfatase-inhibitors used according to the invention, where liquid, can be applied to flexible and absorbent carriers and made available in the form of deodorant or antiperspirant cloths or sponges. As flexible and absorbent carriers in the sense of the invention there are suitable e.g. carriers of textile fibres, collagen or polymeric foam materials. As textile fibres it is possible to use, woven or non-woven, either natural fibres, such as cellulose (cotton, linen), silk, wool, regenerated cellulose (viscose, rayon), cellulose deratives, or synthetic fibres, e.g. polyester, polyacrylonitrile, polyamide or polyolefin fibres, or mixtures of such fibres. The fibres may be processed to form absorbent cotton wadding pads, non-woven materials or woven or knitted materials. Flexible and absorbent polymeric foam materials, e.g. polyurethane foams and polyamide foams, are also suitable substrates. The substrate may have one, two, three or more than three layers, the individual layers consisting of the same or different materials. Each substrate layer may have a homogeneous structure or a non-homogeneous structure with, for example, different zones of different density.

Carrier substrates that are to be regarded as absorbent in the sense of the invention are those which at 20° C. are able to bind at least 10% by weight water, based on the dry weight, by adsorption and/or capillary action. Preferably, however, such carriers are able to bind at least 100% by weight water by adsorption and by capillary action.

The finishing of the carrier substrates is effected by treating or finishing the absorbent, flexible carrier substrates, preferably of textile fibres, collagen or polymeric foamed materials, with the compositions according to the invention and optionally drying. That treatment (finishing) of the carrier substrates can be carried out in accordance with any procedures, e.g. by spraying, immersion and squeezing, impregnation or simply by injecting the composition according to the invention into the carrier substrates.

Also preferred according to the invention is a form of application in aerosol form, the cosmetic composition comprising a propellant selected from propane, butane, isobutane, pentane, isopentane, dimethyl ether, fluorohydrocarbons and fluorochlorohydrocarbons and mixtures thereof.

The following Examples are intended to illustrate the invention, but the invention is not limited thereto.

### EXAMPLES OF FORMULATIONS

Non-aqueous Surfactant-containing Antiperspirant Sticks (Data in Parts by Weight)

| | 1.1 | 1.2 | 1.3 | 1.4 | 1.5 | 1.6 | 1.7 | 1.8 | 1.9 |
|---|---|---|---|---|---|---|---|---|---|
| Silicone oil DC ® | 28 | 28 | 23 | 23 | 23 | 38 | 42 | 47 | 31 |
| 245 | | | | | | | | | |
| Eutanol ® G 16 | 10 | — | — | 15 | 10 | — | 10 | — | 10 |
| Cetiol ® OE | — | 10 | 15 | — | — | — | — | — | — |
| Ucon Fluid ® AP | 5 | 5 | 5 | 5 | 5 | 5 | 5 | 5 | 5 |
| Cutina ® HR | 6 | 6 | 6 | 6 | 6 | 6 | 2 | 5 | 6 |
| Lorol ® C 18 | 20 | 20 | 20 | — | 20 | 20 | — | — | 20 |
| Lanette ® O | — | — | — | 20 | — | — | 10 | 12 | — |
| Eumulgin ® B 3 | 3 | 3 | 3 | 3 | 3 | 3 | 3 | 3 | — |
| Cutina ® E 24 PF | — | — | — | — | 5 | — | — | — | — |
| Aluminium chlorohydrate | 20 | 20 | 20 | 20 | 20 | 20 | 20 | — | — |
| Talcum | 7.995 | 7.9 | 7.9 | 7.9 | 7.5 | 7.5 | 7.995 | 27.9 | 27.5 |
| 4-(2,5-Dimethylphenoxy) | 0.005 | — | — | — | — | — | — | — | — |
| phenol | | | | | | | | | |
| 4-(3-Isopropyl-6- | — | 0.1 | — | — | — | — | — | — | — |
| methylphenoxy)- | | | | | | | | | |
| phenol | | | | | | | | | |
| 4-(2-tert-Butyl-5- | — | — | 0.1 | — | — | — | — | — | — |
| methylphenoxy)- | | | | | | | | | |
| phenol | | | | | | | | | |
| 4-(2-Cyclohexyl-5- | — | — | — | 0.1 | — | — | — | — | — |
| methylphenoxy)- | | | | | | | | | |
| phenol | | | | | | | | | |
| 4-(2-Isopropyl-5- | — | — | — | — | 0.5 | — | — | — | — |
| methylphenoxy)- | | | | | | | | | |
| phenol | | | | | | | | | |
| 4-(3-Isopropyl-5- | — | — | — | — | — | 0.5 | — | — | — |
| methylphenoxy)- | | | | | | | | | |
| phenol | | | | | | | | | |
| 3-(2-Isopropyl- | — | — | — | — | — | — | 0.005 | — | — |
| phenoxy)phenol | | | | | | | | | |
| 3-(2-sec-Butylphenoxy) | — | — | — | — | — | — | — | 0.1 | — |
| phenol | | | | | | | | | |
| 3-Phenoxy-6- | — | — | — | — | — | — | — | — | 0.5 |
| propylphenol | | | | | | | | | |

Sprayable Transluctant Deodorant Microemulsion (Data in % by Weight)

| | 2.1 | 2.2 | 2.3 | 2.4 | 2.5 | 2.6 | 2.7 | 2.8 | 2.9 |
|---|---|---|---|---|---|---|---|---|---|
| Plantaren ® 1200 | 1.71 | 1.71 | — | 1.71 | 1.71 | — | 1.71 | 1.71 | 1.71 |
| Plantaren ® 2000 | 1.14 | 1.39 | 2.40 | 1.14 | 1.39 | 2.40 | 1.14 | 1.39 | 1.39 |
| Glycerol monooleate | 0.71 | 0.71 | — | 0.71 | 0.71 | — | 0.71 | 0.71 | 0.71 |
| Dioctyl ether | 4.00 | 4.00 | 0.09 | 4.00 | 4.00 | 0.09 | 4.00 | 4.00 | 4.00 |
| Octyl dodecanol | 1.00 | 1.00 | 0.02 | 1.00 | 1.00 | 0.02 | 1.00 | 1.00 | 1.00 |
| Perfume oil | 1.00 | 1.00 | 1.00 | 1.00 | 1.00 | 1.00 | 1.00 | 1.00 | 1.00 |
| Aluminium chlorohydrate | 8.00 | 5.00 | 5.00 | — | — | — | 8.00 | 5.00 | 5.00 |
| 1,2-Propylene glycol | 5.00 | 5.00 | — | 5.00 | 5.00 | — | 5.00 | 5.00 | 5.00 |
| Glycerol | — | — | 5.00 | — | — | 5.00 | — | — | — |
| Tocopheryl acetate | 0.6 | 0.6 | 0.6 | 0.6 | 0.6 | 0.6 | 0.6 | 0.6 | 0.6 |
| 4-(2-tert-Butyl-5- | 0.05 | — | — | — | — | — | — | — | — |
| methylphenoxy)phenol | | | | | | | | | |
| 3-Phenoxy-6-(2-methylpropyl) | — | 0.1 | — | — | — | — | — | — | — |
| phenol | | | | | | | | | |
| 3-Phenoxy-6-butyl- | — | — | 0.005 | — | — | — | — | — | — |
| phenolbutylphenol | | | | | | | | | |
| 3-Phenoxy-6-pentylphenol | — | — | — | 0.5 | — | — | — | — | — |
| 3-Phenoxy-6-hexylphenol | — | — | — | — | 0.1 | — | — | — | — |
| 3-Phenoxy-6-(3-methylbutanoyl) | — | — | — | — | — | 0.05 | — | — | — |
| phenol | | | | | | | | | |
| 3-Phenoxy-6-hexanoylphenol | — | — | — | — | — | — | 0.1 | — | — |
| 2-(2-Isopropylphenoxy) | — | — | — | — | — | — | — | 0.1 | — |
| phenol | | | | | | | | | |
| 4-(3-Isopropyl-5- | — | — | — | — | — | — | — | — | 0.005 |
| methylphenoxy)phenol | | | | | | | | | |
| Water | ad 100 | ad 100 | ad 100 | ad 100 | ad 100 | ad 100 | ad 100 | ad 100 | ad 100 |

Soap-containing Deodorant Sticks (Data in % by Weight)

| | 3.1 | 3.2 | 3.3 | 3.4 | 3.5 | 3.6 | 3.7 | 3.8 |
|---|---|---|---|---|---|---|---|---|
| Ethanol | 22.5 | 22.5 | 22.5 | 22.5 | 22.5 | 22.5 | 22.5 | 22.5 |
| Cutina ® FS 45 | 4.4 | 4.4 | 4.4 | 4.4 | 4.4 | 4.4 | 4.4 | 4.4 |
| 1,3-Butanediol | 31.7 | 31.7 | 31.7 | 31.7 | 31.7 | 31.7 | 31.7 | 31.7 |
| 1,2-Propylene glycol | 21.0 | 21.0 | 21.0 | 21.0 | 21.0 | 21.0 | 21.0 | 21.0 |
| Eutanol ® G | 2.0 | 2.0 | 2.0 | 2.0 | 2.0 | 2.0 | 2.0 | 2.0 |
| Aethoxal ® B | 3.0 | 3.0 | 3.0 | 3.0 | 3.0 | 3.0 | 3.0 | 3.0 |
| Cremophor ® RH 455 | 0.05 | 0.05 | 0.05 | 0.05 | 0.05 | 0.05 | 0.05 | 0.05 |
| NaOH 45% | 1.44 | 1.44 | 1.44 | 1.44 | 1.44 | 1.44 | 1.44 | 1.44 |
| Phenoxyethanol | 1.0 | 1.0 | 1.0 | 1.0 | 1.0 | 1.0 | 1.0 | 1.0 |
| Sensiva ® SC 50 | 0.3 | 0.3 | 0.3 | 0.3 | 0.3 | 0.3 | 0.3 | 0.3 |
| Perfume oil | 2.0 | 2.0 | 2.0 | 2.0 | 2.0 | 2.0 | 2.0 | 2.0 |
| 2-(2-tert-Butylphenoxy) | 0.1 | — | — | — | — | — | — | — |
| phenol | | | | | | | | |
| 2-(2-Ethylphenoxy)- | — | 0.005 | — | — | — | — | — | — |
| phenol | | | | | | | | |
| 4-(2,5-Dimethylphenoxy) | — | — | 0.1 | — | — | — | — | — |
| phenol | | | | | | | | |
| 4-(3-Isopropyl-6- | — | — | — | 0.5 | — | — | — | — |
| methylphenoxy)- | | | | | | | | |
| phenol | | | | | | | | |
| 4-(2-tert-Butyl-5- | — | — | — | — | 0.5 | — | — | — |
| methylphenoxy)phenol | | | | | | | | |
| 4-(2-Cyclohexyl-5- | — | — | — | — | — | 0.005 | — | — |
| methylphenoxy)- | | | | | | | | |
| phenol | | | | | | | | |
| 4-(3-Isopropyl-5- | — | — | — | — | — | — | 0.1 | — |
| methylphenoxy)- | | | | | | | | |
| phenol | | | | | | | | |
| 3-(2-Isopropylphenoxy) | — | — | — | — | — | — | — | 0.5 |
| phenol | | | | | | | | |
| Water dist. | ad 100 | ad 100 | ad 100 | ad 100 | ad 100 | ad 100 | ad 100 | ad 100 |

Deodorant in Pump-action Atomiser (Data in % by Weight)

| | 4.1 | 4.2 | 4.3 | 4.4 | 4.5 | 4.6 | 4.7 |
|---|---|---|---|---|---|---|---|
| Ethanol 96%, (DEP | 55.0 | 55.0 | 55.0 | 55.0 | 55.0 | 55.0 | 55.0 |
| denatured) | | | | | | | |
| Triethyl citrate | 3.0 | 3.0 | 3.0 | 3.0 | 3.0 | 3.0 | 3.0 |
| Cremophor ® RH 455 | 0.5 | 0.5 | 0.5 | 0.5 | 0.5 | 0.5 | 0.5 |
| 4-(2-tert-Butyl-5- | 0.5 | — | — | — | — | — | — |
| methylphenoxy) | | | | | | | |
| phenol | | | | | | | |
| 3-Phenoxy-6-propylphenol | — | 0.005 | — | — | — | — | — |
| 3-Phenoxy-6-(2- | — | — | 0.005 | — | — | — | — |
| methylpropyl)phenol | | | | | | | |
| 3-Phenoxy-6-butylphenol | — | — | — | 0.1 | — | — | — |
| 3-Penoxy-6-pentylphenol | — | — | — | — | 0.1 | — | — |
| 3-Phenoxy-6-hexylphenol | — | — | — | — | — | 0.005 | — |
| 3-Phenoxy-6-(3- | — | — | — | — | — | — | 0.1 |
| methylbutanoyl) | | | | | | | |
| phenol | | | | | | | |
| Perfume oil | 1.0 | 1.0 | 1.0 | 1.0 | 1.0 | 1.0 | 1.0 |
| Water | ad 100 | ad 100 | ad 100 | ad 100 | ad 100 | ad 100 | ad 100 |

Non-aqueous Deodorant Spray (Data in % by Weight)

| | 5.1 | 5.2 | 5.3 |
|---|---|---|---|
| 2-Octyl dodecanol | 0.5 | 0.5 | 0.5 |
| Ethanol 99% (DEP denatured) | 38.1 | 39.495 | 38.85 |
| 3-Phenoxy-6-hexanoylphenol | 0.5 | — | 0.5 |
| 3-(2-sec-Butylphenoxy)phenol | — | 0.005 | 0.005 |
| 4-(2-tert-Butyl-5-methylphenoxy)- | 0.1 | — | 0.1 |
| phenol | | | |
| n-Butane | 60 | 60 | 60 |

Antiperspirant Roll-on (Data in % by Weight)

| | 6.1 | 6.2 | 6.3 | 6.4 | 6.5 | 6.6 |
|---|---|---|---|---|---|---|
| Ethanol 96% (DEP denatured) | 30.0 | 30.0 | 30.0 | 30.0 | 30.0 | 30.0 |
| Mergital ® CS 11 | 2.0 | 2.0 | 2.0 | 2.0 | 2.0 | 2.0 |
| Eumulgin ® B 3 | 2.0 | 2.0 | 2.0 | 2.0 | 2.0 | 2.0 |
| Aluminium chlorohydrate | 20.0 | 20.0 | 20.0 | 20.0 | 20.0 | 20.0 |
| Hydroxyethylcellulose | 0.5 | 0.5 | 0.5 | 0.5 | 0.5 | 0.5 |
| 2-(2-Isopropylphenoxy)phenol | 0.1 | — | — | — | — | — |
| 2-(2-sec-Butylphenoxy)phenol | — | 0.005 | — | — | — | — |
| 2-(2-tert-Butylphenoxy)phenol | — | — | 0.1 | — | — | — |
| 3-(2-Isopropylphenoxy)phenol | — | — | — | 0.1 | — | — |
| 3-(2-sec-Butylphenoxy)phenol | — | — | — | — | 0.005 | — |
| 4-(2-tert-Butyl-5- | — | — | — | — | — | 0.1 |
| methylphenoxy)phenol | | | | | | |
| Perfume oil | 0.8 | 0.8 | 0.8 | 0.8 | 0.8 | 0.8 |
| Water | ad 100 | ad 100 | ad 100 | ad 100 | ad 100 | ad 100 |

Antiperspirant Spray of the Suspension Type (Data in % by Weight)

| | 7.1 | 7.2 | 7.3 |
|---|---|---|---|
| DC-245 | 10.0 | 10.0 | 10.0 |
| Isopropyl myristate | 5.0 | 5.0 | 5.0 |
| Aluminium chlorohydrate | 5.0 | 5.0 | 5.0 |
| powder | | | |
| Aerosil ® R 972 | 2.0 | 2.0 | 2.0 |
| 4-(2,5-Dimethylphenoxy)phenol | 0.1 | — | — |
| 4-(3-Isopropyl-6-methylphenoxy) | — | 0.005 | — |
| phenol | | | |
| 4-(2-tert-Butyl-5- | — | — | 0.1 |
| methylphenoxy)phenol | | | |
| n-Butane | 77.9 | 77.995 | 77.9 |

Transparent Antiperspirant Gel (Data in % by Weight)

| | | 8.1 |
|---|---|---|
| Phase 1 | DC-245 | 7.0 |
| | DC-3225 | 10.0 |
| | 4-(2-tert-Butyl-5- | 0.1 |
| | methylphenoxy)phenol | |
| Phase 2 | Chlorhydrol ® | 50.0 |
| | 1,2-Propylene glycol | 16.0 |
| | Water | 16.9 |

With stirring (900 rev/min using a propeller stirrer), Phase 2 is added to Phase 1 in the course of 25 minutes with the aid of a burette. The mixture is then stirred for 30 minutes. A viscous transparent mass having a viscosity of 43750 mPas is obtained.

The mass is then uniformly homogenised for 120 seconds by moving the glass using a shearing head (Ultra Turrax T50 (IKA-Werke), Turraxstab, Step 8 (about 8500 rev/min)). The refractive index is 1.3990 (20° C.).

The following viscosities are measured:

| | | |
|---|---|---|
| t₀ | 61 units | 152500 mPas |
| 73 hours | 76 units | 190000 mPas |
| Day 11 | 68.5 units | 171250 mPas |

Viscosity measurement conditions:

| | |
|---|---|
| Measuring apparatus: | Brookfield RVF using the Helipath |
| Spindle: | TC, 4 revolutions per minute [symbol] factor 2500 per |
| | scale graduation |
| Measured value: | after 60 seconds |
| Temperature: | 20° C. |

Using as the measuring apparatus a Hach 2100 AN IS Turbidometer Ser 99-100000-423 (ISO Method 2027) (Hach), LED measurement 860 nm, a deaerated sample exhibited a turbidity value of 37 NTU at 21° C.

Antiperspirant or Deodorant Cloths

For the embodiment according to the invention in the form of an antiperspirant cloth or deodorant cloth, a single-layer substrate of 100% viscose having a weight per unit area of 50 g/m² is treated with 75 g of Example emulsion 2.1 or 2.2 or 2.3 per square metre or with 75 g of Example solution 4.1 or 4.2, cut into suitably sized cloths and packaged in sachets.

List of Raw Materials Used

| | | |
|---|---|---|
| Aerosil R 972 | Silica dimethyl silylate | Degussa |
| Aethoxal ® B | PPG-5 laureth-5 | Cognis |
| Cetiol ® OE | Dicaprylyl ether | Cognis |
| Chlorhydrol ® | 50% Aluminium chlorohydrate, 50% | Reheis |
| | water | |
| Cremophor ® RH 455 | Hydrogenated castor oil with 40 | BASF |
| | EO, containing propylene glycol | |
| Cutina ® FS 45 | Fatty acid mixture of palmitic and | Cognis |
| | stearic acid | |
| Cutina ® E 24 PF | PEG-20 glyceryl stearate | Cognis |
| Cutina ® HR | Hydrogenated castor oil | Cognis |
| DC ® 245 | Cyclopentasiloxane | Dow Corning |
| DC ® 3225 | Cyclomethicone/dimethicone | Dow Corning |
| | copolyol | |
| Eumulgin ® B 3 | Ceteareth-30 | Cognis |
| Eutanol ® G | 2-Octyl dodecanol | Cognis |
| Eutanol ® G 16 | 2-Hexyl decanol | Cognis |
| Lanette ® O | Cetyl/stearyl alcohol in a ratio of 1:1 | Cognis |
| Lorol ® C 18 | Stearyl alcohol | Cognis |
| Mergital ® CS 11 | Ceteareth-11 | Cognis |
| Plantaren ® 1200 | Lauryl glucoside, | Cognis |
| | about 50% active substance | |
| Plantaren ® 2000 | Decyl glucoside, about 50% active | Cognis |
| | subst. | |
| Sensiva ® SC 50 | 2-Ethylhexylglycerol ether | Schulke & Mayr |
| Ucon Fluid ® AP | PPG-14 butyl ether | Amerchol (Union Carbide) |

Examination of the Inhibitory Action of Arylsulfatase-inhibitors (In Vitro)

The inhibitory action of the inhibitors used according to the invention is tested with reference to a commercially available arylsulfatase (EC 3.1.6.1) obtained from Aerobacter aerogenes. The tests are carried out using sulfatase enzyme assay product No. S-1629 from Sigma in accordance with the information in the Sigma Quality Control Test Procedure data sheet. Ascorbic acid and ATP are used as reference inhibitors.

For evaluation, the arylsulfatase-catalysed formation of p-nitrophenol from the substrate p-nitrophenyl sulfate (pNPS) is monitored by spectrophotometry (λ=420 nm). The 1000 μl reaction solutions, which have been heated to a reaction temperature of 37° C., receive 187 mM Tris/HCl (pH 7.1), 8 mM pNPS and a starting concentration of 0.05 U/ml of arylsulfatase. The unit U of enzyme activity is defined as follows; 1 U of sulfatase hydrolyses 1.0 μmol of pNPS per minute at pH 7.1 and 37° C.

The test procedure has also been disclosed by H. R. Fowler and D. H. Rammler, Biochemistry 3, 230, 1964.

The reaction is started by adding the enzyme and the increase in absorption at λ=420 nm is monitored over 5 minutes, the linear increase in absorption (A) per unit time (t) being a measure of the activity of the enzyme (ΔA/Δt). As reference, the activity of the enzyme in the absence of an inhibitor, (ΔA₁/Δt₁), is set at 100%. Under analogous conditions, the activities in the presence of an inhibitor (ΔA₂/Δt₂) are determined. The inhibitory action of the inhibitor or the reduction in enzyme activity is then calculated in accordance with the formula 100%−(ΔA₂/Δt₂)/(ΔA₁/Δt₁)%.

1-5. (canceled)

6. A method of reducing body odour by means of the inhibition of arylsulfatase on the skin, which comprises applying to said skin a cosmetic deodorant or antiperspirant composition comprising at least one arylsulfatase-inhibiting substance selected from hydroxydiphenyl ethers of general formula 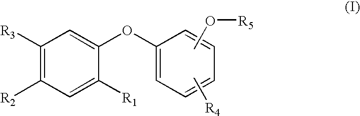
wherein
R₁, R₂ and R₃ independently from each other are hydrogen; hydroxy; C₁-C₂₀alkyl; hydroxy-substituted C₁C₂₀alkyl; C₅-C₇cycloalkyl; C₁-C₂₀alkoxy; C₁-C₆alkylcarbonyl; phenyl; or phenyl-C₁-C₃alkyl;
R₄ hydrogen, C₁-C₂₀alkyl; hydroxy-substitute C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆-alkylcarbonyl; C₁-C₃alkylcarbonyl-C₁-C₃alkyl; phenyl; or phenyl-C₁-C₃alkyl; and
R₅ is hydrogen; C₁-C₂₀alkoxy; or C₁-C₆alkylcarbonyl.

7. A method according to claim 6, wherein the arylsulfatase-inhibiting substance is selected from hydroxydiphenyl ethers of general formula (II) 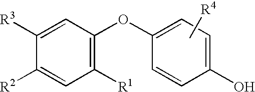
wherein R₁ and R₂ are each independently of the other a hydrogen atom, a hydroxy group or a C₁-C₂₀alkyl, C₅-C₇cycloalkyl, C₁-C₆alkylcarbonyl, C₁-C₂₀alkoxy, phenyl or phenyl-C₁-C₃alkyl group, R₃ is a hydrogen atom or a C₁-C₂₀alkyl or C₁-C₂₀alkoxy group and R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
hydroxydiphenyl ethers of general formula 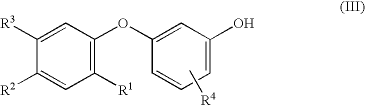
wherein R₂ is a hydrogen atom or a C₁-C₂₀alkyl, hydroxy-substituted C₁-C₂₀alkyl or C₁-C₆alkylcarbonyl group, R₁ and R₃ are each independently of the other a hydrogen atom, a C₁-C₆alkylcarbonyl group or a C₁-C₂₀alkyl group and R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group,
and hydroxydiphenyl ethers of general formula 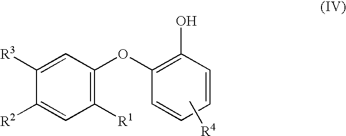
wherein R₁ is a hydrogen atom or a C₁-C₆alkylcarbonyl or C₁-C₂₀alkyl group, R₄ is a hydrogen atom or a C₁-C₂₀alkyl, hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl group and R₂ and R₃ are each independently of the other a hydrogen atom or a C₁-C₆alkylcarbonyl or C₁-C₂₀alkyl group,
is applied to the skin.

8. A method of reducing body odour according to claim 6 wherein the arylsulfatase-inhibiting substance is used gender-specifically in respect of the amount and/or nature thereof.

9. Method of reducing body odour according to claim 6, wherein the arylsulfatase-inhibiting substance is used for reducing body odour in men.
